(19) United States
(12) Patent Application Publication
(10) Pub. No.: US 2007/0275107 A1
(43) Pub. Date: Nov. 29, 2007

(54) Method for the Removal of Aloin, Emodin and/or Isoemodin from Aloe Vera by Treatment with an Oxidase

(76) Inventors: Mark Rusch Gen. Klaas, Neubrandenburg (DE); Andrea Dornbrack, Neubrandenburg (DE); Kurt Beer, Hamburg (DE)

Correspondence Address: NIXON & VANDERHYE, PC, 901 NORTH GLEBE ROAD, 11TH FLOOR, ARLINGTON, VA, 22203, US

(21) Appl. No.: 10/568,769

(22) PCT Filed: Aug. 20, 2004
(86) PCT No.: PCT/EP04/09363

§ 371 Date: Feb. 7, 2007

(30) Foreign Application Priority Data
Aug. 22, 2003 (DE) 10338735.8

(51) Int. Cl. A61K 36/886

(52) U.S. Cl. 424/744

(57) ABSTRACT

The invention relates to a method for the removal of aloin, emodin and/or isoemodin from aloe vera gel and a method for the production of aloe vera gel with less than 5 ppm of aloin, emodin and/or isoemodin by treatment with an oxidase, whereby a manual slicing of the aloe vera leaves and losses associated with conventional production methods are avoided.

The present invention relates to a method of removing aloin, emodin and/or iso-emodin from Aloe Vera gel and to a method of preparing Aloe Vera gel with a content of less than 5 ppm of aloin, emodin and/or iso-emodin, where manual filleting of the Aloe Vera leaves and losses in relation to conventional preparation methods are avoided.

In the Aloe Vera plant family, there are the two species Aloe barbadensis Miller and Aloe aborenscens, both of which are currently exploited commercially. These perennial plants from the family Liliaceae have their origin in Eastern Africa; they are currently grown essentially along the border between the United States and Mexico. Cultivation in Europe is insignificant.

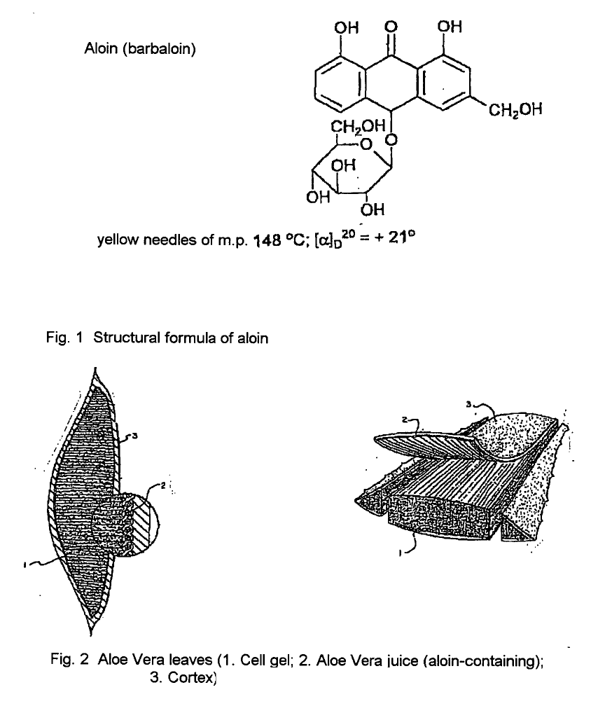

Two products can be obtained from Aloe Vera leaves. The region directly underneath the cortex gives a yellowish juice (a type of latex). This juice contains aloin, a glycosylated anthrachinon derivative, as its main constituent (see FIG. 1). Aloin is a strong laxative which has been used as such for more than 2000 years; nowadays, it is unimportant. The inner tissue of the plant's leaves, which has a high water content, gives the Aloe Vera gel, which is employed in a number of ways in cosmetics and other natural products. Its annual turnover is estimated to be 7 billion [symbol] . This Aloe Vera gel must no longer contain any aloin, or the unglycosylated anthraquinone derivatives emodin or iso-emodin. A content of less than 5 ppm is desired, but not achieved with all products. In the prior art, Aloe Vera gels with aloin contents of less than 20 ppm have hitherto only been achieved by laborious manual filleting of the Aloe Vera leaves. In the prior art, Aloe Vera products with low aloin contents always involve losses some of which are considerable.

The processing into Aloe Vera gel usually involves the following procedure. After washing, the leaves are filleted (FIG. 2) in order to remove the aloin before further processing. As a result of the concave shape of the leaves, their substantial size differences and the structure of the leaf, this filleting step is predominantly carried out manually. This procedure results in losses of 20-60% of the harvested material.

In the past there has been no lack of attempts to peel the Aloe Vera leaves mechanically; however, the aloin concentration which remains in the product is unacceptably high.

It was therefore the object of the present invention to provide a method with which the aloin concentration in products such as the Aloe Vera gel can be lowered substantially and manual filleting can be avoided, without losing an unduly high proportion of harvested material in the process.

This object was solved in accordance with the invention by using oxidases.

Nothing is known from the relevant specialist literature about enzymes or enzyme systems which have a specific aloin-degrading activity; however, in the gastrointestinal tract, aloin is deglucosidated by glucosidases, and the resulting iso-emodin has the actual laxative activity.

A qualitative detection of aloin, emodin and their isomers can be performed particularly successfully by thin-layer chromatography. A suitable quantitative analytical method is in particular photometry, because the compounds in question absorb highly in the visible light range. However, none of the methods allow any conclusions regarding the nature of any degradation products which may have formed. These were analyzed by gas chromatography coupled with mass spectrometry (GC-MS). However, only emodin (which is unglucosidated) can be vaporized without decomposition and is therefore suitable for GC analysis.

Surprisingly, aloin has now been successfully degraded for the first time with the aid of oxidizing enzymes (oxidases), in particular from the classes of the peroxidases (E.C. 1.11.1.7) and laccases (E.C. 1.10.3.2.). As is known, peroxidases act on substrates such as, for example, guaiacol; laccases act on the lignin constituents; it must be considered as surprising that these enzymes accept aloin or emodin as substrates. The oxidant which was preferably used in the laboratory for this purpose was hydrogen peroxide, of which even small amounts can be metered precisely; in the case of larger batches, however, the oxidation can also be accomplished with the aid of atmospheric oxygen.

The degradation of aloin was first detected by thin-layer chromatography in direct comparison with an untreated sample. Moreover, a drastic drop in the typical absorption was observed in the photometer at 328 nm. It has been observed that the degradation of emodin is completely analogous. Moreover, after emodin has been subjected to oxidative degradation, a gas chromatograph shows four new signals at a markedly reduced retention time. The evaluation of the mass spectra demonstrated that the signals were salicylic acid derivatives.

The invention therefore relates to a process for removing aloin, emodin and/or iso-emodin from Aloe Vera gel where the gel is brought into contact with an oxidase under conditions which are suitable for the enzymatic activity.

Thus, there is furthermore provided a process of preparing Aloe Vera gel with a content of less than 5 ppm of aloin, emodin and/or iso-emodin without manual filleting, where the gel is brought into contact with an oxidase under conditions which are suitable for the enzymatic activity. If appropriate, the oxidase is removed from the gel after the reaction has taken place, for example in order to re-use the enzyme.

According to a particular embodiment of the invention, the oxidase is a peroxidase, preferably peroxidase E.C. 1.11.1.7 from Glycine max., or a laccase, preferably oxidase E.C. 1.10.3.2 from Rhus vernificera.

The oxidase can be present in isolated or purified form or else in the form of an extract from a natural substance. In the method according to the invention, the oxidant used is hydrogen peroxide or (atmospheric) oxygen.

The enzymatic reaction is advantageously carried out in an aqueous suspension or solution of the Aloe Vera gel. In accordance with a particular embodiment of the invention, the suspension or solution is buffered to the pH value which is the activity maximum of the enzyme.

The Aloe Vera gel is preferably obtainable by an abovementioned process, or process described in the examples section, respectively. However, it is clear to a person skilled in the art that modifications of the process according to the invention are possible (such as, for example, variations of the reaction conditions, solvents, enzymes and the like), which essentially lead to the same or an improved result, which is the degradation of aloin, emodin and/or iso-emodin in Aloe Vera gel.

The particular advantage of the present invention is that laborious manual cutting up of the Aloe Vera leaves is not required and that therefore losses of the raw material are avoided and a very pure product is obtained.

Aloe Vera gel contains a large number of organic valuable constituents. Approximately 160 such constituents are known; the most important is what is known as alloverose, a pentasaccharide. Alloverose is virtually completely eliminated for example by the known unselective adsorption onto activated charcoal, with the aid of which it is also possible to reduce the aloin content (cf., for example, U.S. Pat. No. 5,356,811), and the dermatological value of such Aloe Vera gels is thus distinctively reduced. In contrast, the method according to the invention for the biocatalytic removal of aloin does not attack alloverose.

The invention is illustrated in greater detail by the examples which follow; they, however, do not limitat the invention described.

### EXAMPLES

### Example 1

0.5 mg of peroxidase (E.C. 1.11.1.7; Glycine max.) and 8 μl of a 3% hydrogen peroxide solution are added to 3 ml of an ethanolic solution of 1000 ppm of aloin in a 1 cm PMMA cuvette. Within 2 hours, the absorbance in the photometer at 328 nm drops from 1.589 to 0.538. After 12 hours, the thin-layer chromatogram no longer reveals an aloin signal.

### Example 2

0.5 mg of peroxidase and 8 μl of a 3% hydrogen peroxide solution are added to 3 ml of a solution of 200 ppm of emodin in tetrahydrofuran in a 1 cm quartz glass cuvette. Within 2 hours, the absorbance in the photometer at 328 nm drops from 2.646 to 0.709. After 12 hours, the thin-layer chromatogram no longer reveals an emodin signal. The gas chromatogram reveals a new signal at 13.6 min (emodin 19.4 min). The mass spectrum shows dimer signals of the degradation products at 148+133 u.

### Example 3

Example 1 is repeated with an aqueous suspension of 1000 ppm of aloin. Again, the absorbance in the photometer at 328 nm drops, and the thin-layer chromatogram no longer reveals an aloin signal after 12 hours.

### Example 4

2 mg of peroxidase are added to 4 ml of a 2.5% solution of emodin in ethanol/water (20/80 v/v) and a gentle stream of compressed air is subsequently passed in over two hours, using a Pasteur pipette. During this process, the color of the solution changes from deep yellow to dark red. A fraction with the characteristics described in example 2 can be separated by column chromatography.

### Example 5

Example 1 is repeated with 0.5 mg of a laccase (E.C. 1.10.3.2.; Rhus vernificera) whose activity had previously been ensured with syringaldazin. A suspension of 1000 ppm of aloin in phosphate buffer (pH value 6.5) with addition of 5% by volume of ethanol is used. Again, the absorbance in the photometer at 328 nm drops, and the thin-layer chromatogram no longer reveals an aloin signal after 12 hours.

### Example 6

Example 1 is repeated with an Aloe Vera gel (1:100) with an aloin content of 250 ppm (noncommerical product). Again, the absorbance in the photometer at 328 nm drops, and the thin-layer chromatogram no longer reveals an aloin signal after 12 hours.

### Example 7

Example 6 is repeated with an Aloe Vera gel (1:100) with a known alloverose content. The above-described treatment does not reduce the alloverose content. If, in contrast, the Aloe Vera gel is filtered through activated charcoal, alloverose is no longer detectable thereafter.

1. A process for removing aloin, emodin and/or iso-emodin from Aloe Vera gel, characterized in that the gel is brought into contact with an oxidase under conditions which are suitable for the enzymatic activity.

2. A process for preparing Aloe Vera gel with a content of less than 5 ppm of aloin, emodin and/or iso-emodin, characterized in that the gel is brought into contact with an oxidase under conditions which are suitable for the enzymatic activity.

3. The process according to claim 1, characterized in that the oxidase is removed from the gel after the reaction has taken place.

4. The process according to claim 1, characterized in that the oxidase is a peroxidase or a laccase.

5. The process according to claim 4, characterized in that the peroxidase is peroxidase E.C. 1.11.1.7 from Glycine max.

6. The process according to claim 4, characterized in that the oxidase is oxidase E.C. 1.10.3.2 from Rhus vernificera.

7. The process according to claim 1, characterized in that the oxidase is present in the form of an extract from a natural substance.

8. The process according to claim 1, characterized in that the oxidant used is hydrogen peroxide or (atmospheric) oxygen.

9. The process according to claim 1, characterized in that the enzymatic reaction is carried out in an aqueous suspension or solution of the Aloe Vera gel.

10. The process according to in claim 9, characterized in that the suspension or solution is buffered.
